(19) United States
(12) Patent Application Publication
(10) Pub. No.: US 2025/0378911 A1
(43) Pub. Date: Dec. 11, 2025

(54) SYSTEM AND METHOD FOR EXPLAINABLE OPTIMIZATION OF PROTEIN SEQUENCE USING INVERSE FOLDING MODEL

(71) Applicant: Quantiphi, Inc., Marlborough, MA (US)

(72) Inventors: Dagnachew Birru, Marlborough, MA (US); Tehemton K Khairabadi, Mumbai (IN); Vishal Pagidipally, Toronto (CA)

(21) Appl. No.: 19/309,027

(22) Filed: Aug. 25, 2025

(51) Int. Cl. G16B 30/10; G06N 3/047; G16B 15/20; G16B 40/20

(52) U.S. Cl. CPC G16B 30/10; G06N 3/047; G16B 15/20; G16B 40/20

(57) ABSTRACT

A method (400) and system (100) for explainable optimization of protein sequence is disclosed. The method (400) includes initializing Position-Specific Scoring Matrix (PSSM) based on probability distribution of the inverse folding model. The method (400) may include generating plurality of protein sequences by sampling from an inverse folding model. The method (400) may further include predicting target property value for each of protein sequences using predictor models. The method (400) further includes computing delta value for each protein sequence by subtracting average predicted target property value across plurality of protein sequences from predicted value for each protein sequence. Further, the method (400) includes determining attribution scores for each amino acid in protein sequence using explainable AI. The method (400) further includes computing position-wise amino acid frequency distribution from protein sequences. The method (400) further includes updating PSSM by combining scaled attribution scores and scaled position-wise amino acid frequency distribution.

### FIELD OF THE INVENTION

The present disclosure relates to chemical compounds, and more specifically to a system and method for explainable optimization of protein sequence using inverse folding model.

### BACKGROUND OF THE INVENTION

Protein engineering involves modifying protein sequences to improve their functional properties for applications in therapeutics, industrial enzymes, and other biochemical processes. A primary challenge in protein engineering is the vast size of the protein sequence search space. For example, a protein with 50 amino acid positions, each capable of being one of 20 naturally occurring amino acids, results in a search space of 20\hat{ }50 sequences, making exhaustive sampling computationally infeasible.

Conventional techniques for protein optimization rely on strategies such as oversampling and Reinforcement Learning (RL) based methods. Oversampling may involve generating a large number of protein sequences from a generative model and filtering them based on predicted properties, often requiring millions of sequences to identify high-performing candidates. The conventional approach is computationally expensive and inefficient due to the low probability of sampling optimal sequences from the vast search space. Further, the RL-based methods treat the generative model as a policy within an RL framework, fine-tuning the model's weights to favour sequences with desired properties. However, the conventional techniques often suffer from catastrophic forgetting, where the model loses its ability to generate structurally valid sequences as it is trained to optimize for the target property. To mitigate this, the RL framework approach may incorporate folding accuracy as part of the reward function, but this requires computationally intensive structural validation for each sequence, significantly slowing the optimization process.

Additionally, conventional techniques often lack interpretability, operating as black-box systems where the rationale behind sequence selection is not transparent. The lack of explainability hinders rational design and limits the ability to target specific regions or properties of the protein. Furthermore, the conventional approaches struggle to balance exploration of the protein sequence space with exploitation of known high-performing regions, often converging to local optima rather than global solutions.

Therefore, there is a need for a method and system that navigates the protein sequence search space, reduces the computational burden of sampling, maintains structural accuracy, and provides interpretable insights into the optimization process, enabling faster and more rational design of proteins with enhanced properties.

### SUMMARY

The following embodiments presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Some example embodiments disclosed herein provide computer-implemented method for explainable optimization of protein sequence using inverse folding model, the method may include initializing a Position-Specific Scoring Matrix (PSSM) based on a probability distribution of the inverse folding model. The inverse folding model generates protein sequences from a target protein structure. The method may further include generating a plurality of protein sequences by sampling from the inverse folding model. The sampling is biased by applying the PSSM to the inverse folding model's output probabilities. The method may further include predicting a target property value for each of the plurality of protein sequences using a predictor model. The method may further include computing a delta value for each protein sequence by subtracting an average predicted target property value across the plurality of protein sequences from the predicted value for each protein sequence. Further, the method may include determining token-level attribution scores for each amino acid in each protein sequence using an explainable AI framework. The attribution scores indicates a contribution of each amino acid to the predicted target property value. Further, the method include computing a position-wise amino acid frequency distribution from the plurality of protein sequences. The frequency distribution is scaled by the delta value of each protein sequence to emphasize frequencies in the protein sequences with higher predicted target property value. The method may include updating the PSSM by combining the scaled token-level attribution scores and the scaled position-wise amino acid frequency distribution. The updating is performed using a normalization function to maintain the PSSM as a probability distribution.

According to some example embodiments, the inverse folding model includes a graph neural network-based model comprising ProteinMPNN and HyperMPNN.

According to some example embodiments, the explainable AI framework is selected from the group consisting of Integrated Gradients, SHapley Additive explanations (SHAP), Local Interpretable Model-agnostic Explanations (LIME), and attention-based techniques.

According to some example embodiments, the predictor model is configured to predict one or more of a thermostability, a melting temperature, a solubility, a catalytic activity, and a binding affinity of the protein.

According to some example embodiments, updating the PSSM further includes applying a learning rate to stabilize the combination of the scaled token-level attribution scores and the scaled position-wise amino acid frequency distribution.

According to some example embodiments, the method further includes applying a weight factor to the PSSM. The weight factor control a degree of bias applied to the inverse folding model's output probabilities.

According to some example embodiments, the weight factor is dynamically adjusted using a scheduler to balance exploration and exploitation, wherein the scheduler being selected from the group consisting of a cosine scheduler, a fixed interval scheduler, and a reinforcement learning policy network.

According to some example embodiments, the method further includes masking chains in the protein sequences to optimize only targeted regions of the protein. The PSSM is updated only for the masked regions.

Some example embodiments disclosed herein provide a computer-implemented system for explainable optimization of protein sequence using inverse folding model. The computer-implemented system includes one or more computer processors, one or more computer readable memories, one or more computer readable storage devices, and program instructions stored on the one or more computer readable storage devices for execution by the one or more computer processors via the one or more computer readable memories. The program instructions includes initializing a Position-Specific Scoring Matrix (PSSM) based on a probability distribution of the inverse folding model. The inverse folding model generates protein sequences from a target protein structure. Further, the program instructions includes generating a plurality of protein sequences by sampling from the inverse folding model. The sampling is biased by applying the PSSM to the inverse folding model's output probabilities. The program instructions includes predicting a target property value for each of the plurality of protein sequences using a predictor model. Further, the program instructions includes computing a delta value for each protein sequence by subtracting an average predicted target property value across the plurality of protein sequences from the predicted value for each protein sequence. The program instructions includes determining token-level attribution scores for each amino acid in each protein sequence using an explainable AI framework. The attribution scores indicates a contribution of each amino acid to the predicted target property value. Further, the program instructions includes computing a position-wise amino acid frequency distribution from the plurality of protein sequences. The frequency distribution is scaled by the delta value of each protein sequence to emphasize frequencies in the protein sequences with higher predicted target property value. The program instructions includes updating the PSSM by combining the scaled token-level attribution scores and the scaled position-wise amino acid frequency distribution. The updating is performed using a normalization function to maintain the PSSM as a probability distribution.

Some example embodiments disclosed herein provide a non-transitory computer readable medium having stored thereon computer executable instruction which when executed by one or more processors, cause the one or more processors to carry out operations for explainable optimization of protein sequence using inverse folding model, the operations includes initializing a Position-Specific Scoring Matrix (PSSM) based on a probability distribution of the inverse folding model. The inverse folding model generates protein sequences from a target protein structure. Further, the operations includes generating a plurality of protein sequences by sampling from the inverse folding model. The sampling is biased by applying the PSSM to the inverse folding model's output probabilities. The operations includes predicting a target property value for each of the plurality of protein sequences using a predictor model. Further, the operations includes computing a delta value for each protein sequence by subtracting an average predicted target property value across the plurality of protein sequences from the predicted value for each protein sequence. The operation may include determining token-level attribution scores for each amino acid in each protein sequence using an explainable AI framework. The attribution scores indicates a contribution of each amino acid to the predicted target property value. Further, the operations may include computing a position-wise amino acid frequency distribution from the plurality of protein sequences. The frequency distribution is scaled by the delta value of each protein sequence to emphasize frequencies in the protein sequences with higher predicted target property value. The operations may further include updating the PSSM by combining the scaled token-level attribution scores and the scaled position-wise amino acid frequency distribution. The updating is performed using a normalization function to maintain the PSSM as a probability distribution.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present invention will become apparent upon consideration of the following detailed description of embodiments thereof, especially when taken in conjunction with the accompanying drawings, and wherein:

FIG. 1 is a block diagram of an environment of a system for explainable optimization of protein sequence using inverse folding model, in accordance with an example embodiment.

FIG. 2 is a block diagram illustrating various modules within a memory of a computing device configured for explainable optimization of protein sequence using inverse folding model, in accordance with an example embodiment.

FIG. 3 illustrates a block diagram of a system architecture for explainable optimization of protein sequence using inverse folding model, in accordance with an example embodiment.

FIG. 4 illustrates a flow diagram of a method for explainable optimization of protein sequence using inverse folding model, in accordance with an example embodiment.

FIG. 5 is a block diagram of an exemplary computer system for implementing embodiments consistent with the present disclosure.

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth in order to provide a thorough understanding of the present invention. It will be apparent, however, to one skilled in the art that the present invention can be practiced without these specific details. In other instances, systems, apparatuses, and methods are shown in block diagram form only in order to avoid obscuring the present invention.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “Exploration scheduler” may refer to a mechanism, implemented as either a policy network or a fixed schedule, that dynamically controls the balance between exploration and exploitation during the optimization of protein sequences. The exploration scheduler determines a weight parameter, ranging from 0 to 1, which governs the strength of a learned bias, such as a Position-Specific Scoring Matrix (PSSM), applied to the output probabilities of an inverse folding model.

The term “Position-Specific Scoring Matrix (PSSM)” may refer to a weight matrix used to bias the output probabilities of the inverse folding model during protein sequence generation. The PSSM encodes a probability distribution over amino acids for each position in a protein sequence, initialized either randomly or based on the prior distribution of the inverse folding model.

The term “Protein Sequence” may be used to refer to a linear arrangement of amino acids that defines the primary structure of a protein. The protein sequence may be generated by an inverse folding model based on a given protein backbone structure, represented as a string of amino acid residues, each selected from a set of naturally occurring or modified amino acids.

The term “Inverse folding model” may refer to a computational model, typically implemented as a machine learning model such as a graph neural network, that generates protein sequences conditioned on a given protein backbone structure. The inverse folding model learns a conditional probability distribution over amino acid sequences that are predicted to fold into the provided structure.

The term “Thermostability” may refer to an ability of a protein to maintain its structural integrity and functional activity at elevated temperatures. The thermostability is a target property for optimization, measured as the melting temperature (Tm) at which a protein denatures, with higher melting temperatures indicating greater stability.

The term “Amino acid” may refer to an organic molecule that serves as a building block of proteins, characterized by an amino group, a carboxyl group, and a variable side chain that determines its chemical properties. The amino acid is a single residue within a protein sequence, selected from naturally occurring amino acids or modified variants, which is evaluated and optimized for its contribution to a target property, such as thermostability, through computational methods involving sequence generation and analysis.

The term “Explainable Artificial Intelligence (AI)” may refer to a set of techniques and frameworks designed to provide interpretable insights into the decision-making processes of machine learning models. The explainable AI is used to determine token-level attribution scores that indicate the contribution of individual amino acids in a protein sequence to a predicted property, such as thermostability.

The term “Attribution scores” may refer to a numerical value generated by the explainable AI framework that quantify the contribution of each amino acid (token) in a protein sequence to the predicted value of a target property, such as thermostability. The attribution scores indicate whether an amino acid positively or negatively influences the prediction, with higher scores reflecting a stronger positive contribution and lower or negative scores indicating a detrimental effect.

The term “module” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

### End of Definitions

As described earlier, the vast protein sequence search space (e.g., 20\hat{ }50 possible sequences for a 50-amino-acid protein) makes exhaustive sampling computationally infeasible. Existing methods, such as oversampling or reinforcement learning (RL)-based fine-tuning of generative models, are inefficient, requiring millions of sequences or extensive computational resources. The present disclosure provides a computer-implemented method for efficiently optimizing protein sequences using an inverse folding model, such as a graph neural network, Large Language Model (LLM), and similar structure to sequence method to generate sequences from a given protein backbone. The present disclosure employs a Position-Specific Scoring Matrix (PSSM) to bias the inverse folding model's sampling toward sequences with enhanced target properties (e.g., thermostability). The PSSM is iteratively refined by combining causal insights from an explainable AI framework (e.g., Integrated Gradients), which identifies amino acid contributions to the target property, and correlational data from position-wise amino acid frequency distributions, scaled by performance relative to the batch mean. The inverse folding model weights remain fixed to preserve structural accuracy, avoiding catastrophic forgetting. Further, a tuneable exploration scheduler balances exploration and exploitation to prevent convergence to local optima. The present disclosure achieves faster convergence, greater interpretability, and reduced computational demands compared to existing g techniques, enabling rational and targeted protein design for applications in biotechnology and therapeutics.

Embodiments of the present disclosure may provide a method, a system, and a computer program product for explainable optimization of protein sequence using inverse folding model. The method, the system, and the computer program product optimize the protein sequences in such an improved manner are described with reference to FIG. 1 to FIG. 5 as detailed below.

FIG. 1 illustrates a block diagram of an environment of a system 100 for explainable optimization of protein sequence using inverse folding model, in accordance with an example embodiment. The system 100 is designed to facilitate explainable optimization of protein sequence using inverse folding model, such as graph neural networks. The system 100 includes a computing device 102 and an external device 108. The computing device 102 may be communicatively coupled with the external device 108 via a communication network 110. Examples of the computing device 102 may include, but are not limited to, a server, a desktop, a laptop, a notebook, a tablet, a smartphone, a mobile phone, an application server, or the like.

The communication network 110 may be wired, wireless, or any combination of wired and wireless communication networks, such as cellular, Wi-Fi, internet, local area networks, or the like. In one embodiment, the communication network 110 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

The computing device 102 may include a memory 106, and a processor 104. The term “memory” used herein may refer to any computer-readable storage medium, for example, volatile memory, random access memory (RAM), non-volatile memory, read only memory (ROM), or flash memory. The memory 106 may include a Random-Access Memory (RAM), a Read-Only Memory (ROM), a Complementary Metal Oxide Semiconductor Memory (CMOS), a magnetic surface memory, a Hard Disk Dri.ve (HDD), a floppy disk, a magnetic tape, a disc (CD-ROM, DVD-ROM, etc.), a USB Flash Drive (UFD), or the like, or any combination thereof.

The term “processor” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

The processor 104 may retrieve computer program code instructions that may be stored in the memory 106 for execution of the computer program code instructions. The processor 104 may be embodied in a number of different ways. For example, the processor 104 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 104 may include one or more processing cores configured to perform independently. A multi-core processor may enable multiprocessing within a single physical package. Additionally, or alternatively, the processor 104 may include one or more processors configured in tandem via the bus to enable independent execution of instructions, pipelining, and/or multithreading.

Additionally, or alternatively, the processor 104 may include one or more processors capable of processing large volumes of workloads and operations to provide support for big data analysis. In an example embodiment, the processor 104 may be in communication with a memory 106 via a bus for passing information among components of the system 100.

The memory 106 may be non-transitory and may include, for example, one or more volatile and/or non-volatile memories. In other words, for example, the memory 106 may be an electronic storage device (for example, a computer readable storage medium) comprising gates configured to store data (for example, bits) that may be retrievable by a machine (for example, a computing device like the processor 104). The memory 106 may be configured to store information, data, contents, applications, instructions, or the like, for enabling the apparatus to carry out various functions in accordance with an example embodiment of the present disclosure. For example, the memory 106 may be configured to buffer input data for processing by the processor 104.

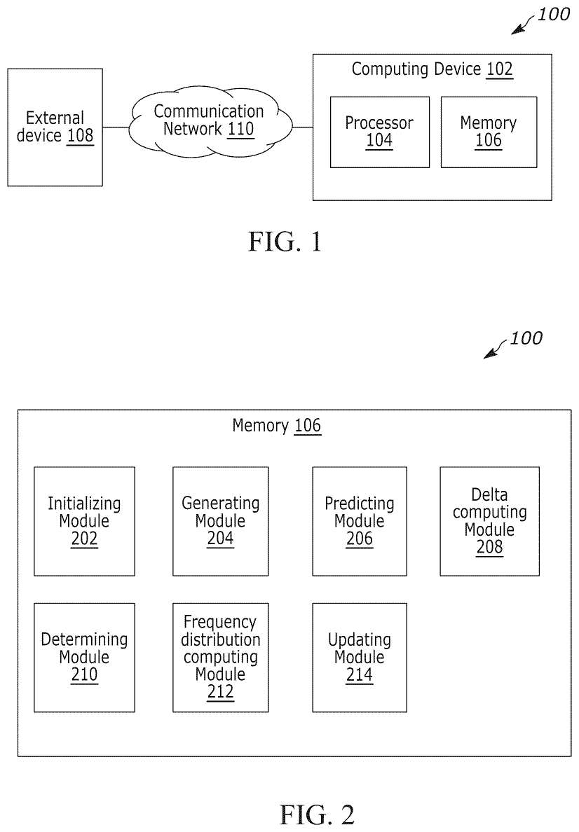

The computing device 102 may be capable of optimize the protein sequence using an inverse folding model. The memory 106 may store instructions that, when executed by the processor 104, cause the computing device 102 to perform one or more operations of the present disclosure which will be described in greater detail in conjunction with FIG. 2. The computing device 102 may be configured to initialize the PSSM based on a probability distribution of the inverse folding model. The inverse folding model generates protein sequences from a target protein structure. Further, the computing device 102 may be configured to generate a plurality of protein sequences by sampling from the inverse folding model. The sampling is biased by applying the PSSM to the inverse folding model's output probabilities. The computing device 102 may further predict a target property value for each of the plurality of protein sequences using a predictor model. Further, the computing device 102 may be configured to compute a delta value for each protein sequence by subtracting an average predicted target property value across the plurality of protein sequences from the predicted value for each protein sequence. The computing device 102 may determine token-level attribution scores for each amino acid in each protein sequence using an explainable AI framework. The attribution scores indicate a contribution of each amino acid to the predicted target property value. Further, the computing device 102 may be configured to compute a position-wise amino acid frequency distribution from the plurality of protein sequences. The frequency distribution is scaled by the delta value of each protein sequence to emphasize frequencies in the protein sequences with higher predicted target property value. Further, the computing device 102 may update the PSSM by combining the scaled token-level attribution scores and the scaled position-wise amino acid frequency distribution. The updating is performed using a normalization function to maintain the PSSM as a probability distribution.

The external devices 108 may refer to various hardware and software tools that may be integrated with the system 100 to enhance its functionality. The complete process followed by the system 100 is explained in detail in conjunction with FIG. 2 to FIG. 5.

FIG. 2 illustrates a block diagram 200 illustrating various modules within the memory 106 of the computing device 102 configured for explainable optimization of protein sequence using inverse folding model, in accordance with an example embodiment. The memory 106 may include an initializing module 202, a generating module 204, a predicting module 206, a delta computing module 208, a determining module 210, a frequency distribution computing module 212, and an updating module 214.

In an embodiment, the initializing module 202 may be configured to initialize a Position-Specific Scoring Matrix (PSSM) based on a probability distribution of the inverse folding model or generated from a database/dataset of other similar proteins. The inverse folding model generates protein sequences from a target protein structure. The inverse folding model comprises a graph neural network-based model comprising ProteinMPNN and HyperMPNN. The PSSM may be a matrix where each row corresponds to a position in the protein sequence, and each column represents a possible amino acid, with entries indicating the probability or weight of selecting a specific amino acid at that position. The inverse folding model is trained on extensive protein structure datasets to learn the conditional probability distribution of amino acid sequences that correspond to a specific three-dimensional structure. In an example, the ProteinMPNN takes geometric and topological features of the protein backbone as input and outputs a probability distribution over possible amino acids for each position. The HyperMPNN may be fine-tuned to bias toward sequences with enhanced properties such as higher thermostability.

The initializing module 202 may include applying a weight factor to the PSSM. The weight factor control a degree of bias applied to the inverse folding model's output probabilities. The weight factor is dynamically adjusted using a scheduler to balance exploration and exploitation. The scheduler being selected from the group consisting of a cosine scheduler, a fixed interval scheduler, and a reinforcement learning policy network. The inverse folding model is designed to generate protein sequences conditioned on a target protein backbone structure, effectively solving the inverse folding problem by learning a conditional probability distribution over amino acid sequences that are predicted to fold into the specified structure. The initializing module 202 establishes the PSSM, a matrix that encodes a probability distribution over amino acids for each position in the protein sequence, either by adopting the prior probability distribution of the inverse folding model or by initializing it randomly to maintain broad coverage while preserving structural accuracy. The initialization ensures that the PSSM starts with a baseline that reflects the model's learned sequence-structure relationships, for subsequent refinement to bias sampling toward sequences with enhanced target properties, such as thermostability.

In an embodiment, the initializing module 202 applies the weight factor to the PSSM, which controls the degree of bias applied to the inverse folding model's output probabilities during sequence generation. The weight factor may range from 0 to 1 which modulates the influence of the PSSM on the model's sampling behaviour. A weight factor closer to 0 reduces the PSSM's influence, allowing the inverse folding model to rely more on the intrinsic probability distribution for broader exploration of the protein sequence space. Conversely, a weight factor closer to 1 amplifies the PSSM's effect, emphasizing exploitation of high-performing sequence regions identified through prior iterations. The weight factor is dynamically adjusted using a scheduler to balance exploration and exploitation, preventing the optimization process from converging prematurely to local optima and enabling the discovery of globally optimal sequences. Further, the scheduler is selected from a group consisting of a cosine scheduler, which periodically varies the weight factor following a cosine function to alternate between exploration and exploitation, a fixed interval scheduler, which adjusts the weight factor at predefined intervals, and a reinforcement learning policy network, which employs algorithms such as Proximal Policy Optimization (PPO) or Advantage Actor-Critic (A2C) to learn optimal exploration-exploitation strategies over time based on feedback from the optimization process. The dynamic adjustment enhances the flexibility and efficiency, allowing to adaptively navigate the vast protein sequence space while targeting sequences with desired properties.

In an embodiment, the generating module 204 may be configured to generate a plurality of protein sequences by sampling from the inverse folding model. The sampling is biased by applying the PSSM to the inverse folding model's output probabilities. The inverse folding model may take a target protein backbone structure as input and produces a probability distribution over amino acids for each position in the sequence, reflecting the likelihood of each amino acid forming a sequence that folds into the specified structure. By applying the PSSM to the inverse folding model output probabilities, the generating module 204 modifies the sampling distribution to favour protein sequences that are likely to exhibit enhanced target properties, such as thermostability, solubility, or binding affinity. The biasing is achieved by multiplying the PSSM's weights with the inverse folding model output log probabilities, effectively increasing the likelihood of selecting amino acids that align with the desired property while maintaining the structural integrity of the generated protein sequences. For each position in the protein sequence, the inverse folding model outputs a probability distribution over possible amino acids (e.g., 20 naturally occurring amino acids), which represents the likelihood of each amino acid contributing to a sequence that matches the input target protein structure. The inverse folding model's weights may remain fixed during the optimization process to prevent catastrophic forgetting, ensuring that the structural accuracy learned during pre-training is preserved.

In an embodiment, the predicting module 206 may be configured to predict a target property value for each of the plurality of protein sequences using a predictor model. The predictor model is configured to predict one or more of a thermostability, a melting temperature, a solubility, a catalytic activity, and a binding affinity of the protein. The predictor model may be a computational model, such as a regression model, designed to evaluate protein sequences and output quantitative predictions for one or more target properties. For each protein sequence generated by the generating module 204, the predicting module 206 processes the protein sequence to produce a numerical value representing the predicted performance of the protein sequence with respect to the target property. For example, in the case of thermostability, the predictor model may output a melting temperature (Tm) in degrees Celsius, indicating the temperature at which the protein is expected to denature. The predictor model may directly analyse the amino acid sequence or, first predict a three-dimensional structure from the protein sequence and then evaluate the target property. The predicted target property values serve as the basis for subsequent optimization steps, enabling the system 100 to identify protein sequences with enhanced properties and guide the refinement of the PSSM to bias future sampling toward high-performing sequences. The predicted target property values are critical for assessing which protein sequences are likely to exhibit the desired property and for computing metrics (e.g., Delta T) that drive the iterative refinement of the PSSM. The predicting module's 206 output enables the computing system 102 to prioritize protein sequences that perform above the batch average of all the generated protein sequences, focusing the optimization process on high-performing regions of the protein sequence space. In an example, for each of the “N” protein sequences generated by the generating module 206, the predicting module 206 processes the protein sequences through the predictor model to generate a predicted value for the target property. For instance, in the case of thermostability, each protein sequence is input into a model like TemBERTure, which outputs a melting temperature such as, 75° C., 80° C., and 45° C.

In an embodiment, the delta computing module 208 may be configured to compute a delta value for each protein sequence by subtracting an average predicted target property value across the plurality of protein sequences from the predicted value for each protein sequence. The delta computing module 208 plays a pivotal role in the protein optimization process by quantifying the relative performance of each protein sequence with respect to a target property. The delta computing module 208 first calculates the mean of the predicted property values across the batch of protein sequences, representing a baseline performance for the batch. For each protein sequence, the delta computing module 208 then computes the delta value by subtracting the mean predicted target property value from the protein sequence's individual predicted target property value, resulting in a positive delta for protein sequences performing above the average and a negative delta for the protein sequences performing below the average. The delta values serve as a critical metric for identifying high-performing sequences and guiding subsequent optimization steps, such as refining the PSSM, by highlighting which protein sequences contribute positively or negatively to the desired property. In an example, for an average target property value of the batch is 75° C., a positive delta T such as 5° C. indicates that the protein sequence contributes positively to the target property and a negative delta T such as −5° C. indicates that the protein sequence contributes negatively to the target property, marking the associated protein sequence as less desirable for optimization.

In an embodiment, the determining module 210 may be configured to determine token-level attribution scores for each amino acid in each protein sequence using an explainable AI framework. The attribution scores indicates a contribution of each amino acid to the predicted target property value. The explainable AI framework is selected from the group consisting of Integrated Gradients, SHapley Additive explanations (SHAP), Local Interpretable Model-agnostic Explanations (LIME), and attention-based techniques. The token-level attribution scores indicate whether an amino acid “positively or negatively” influences the predicted property value, with higher attribution scores reflecting a positive contribution such as increasing thermostability and lower or negative scores indicating a detrimental effect such as reducing thermostability. The determining module 210 is responsible for providing interpretable insights into the contributions of individual amino acids to the predicted target property. The determining module 210 employs an explainable AI framework to generate token-level attribution scores, which are used to identify which amino acids in a sequence are driving the predictor model's output. The explainable AI takes the protein sequence and the predicted property value (e.g., 75° C. for thermostability) as input and outputs a set of attribution scores for each amino acid. The explainable AI may provide an analogy where words like “extremely” and “good” receive high attribution scores for positive sentiment, similarly, in protein optimization, amino acids that enhance the target property receive high scores, while those that detract receive low or negative scores.

The token-level attribution scores may be numerical values assigned to each amino acid in the protein sequence, reflecting the influence on the predictor model's output for the target property. For the protein sequence predicted to have a specific property value such as a melting temperature of 75° C., the determining module 210 analyses the protein sequence to determine which amino acids contribute positively such as increasing the predicted temperature and which contribute negatively. For example, in the protein sequence predicted to be stable at 75° C., certain amino acids may receive high positive scores for enhancing thermostability, while others receive low or negative scores for reducing it. Similarly, for a sequence with a lower predicted value (e.g., 45° C.), the determining module 210 identifies amino acids that contribute to the lower performance, which are then penalized in subsequent optimization steps. In some embodiments, the attribution scores may be normalized or scaled to ensure consistency across the protein sequences and iterations.

In an embodiment, the frequency distribution computing module 212 may be configured to compute a position-wise amino acid frequency distribution from the plurality of protein sequences. The frequency distribution is scaled by the delta value of each protein sequence to emphasize frequencies in the protein sequences with higher predicted target property value. The plurality of protein sequences evaluated for a target property (e.g., thermostability) by the predicting module 206. The frequency distribution computing module 212 analyses the protein sequences to determine the frequency of each amino acid at each position across the protein sequence set, creating a position-wise amino acid count matrix. The matrix is then scaled by the delta value of each protein sequence, computed by a delta computing module 208. By scaling the matrix with delta values, the frequency distribution computing module 212 prioritizes amino acids associated with high-performing sequences, enhancing the ability to refine the PSSM and bias sampling toward sequences with improved target properties (e.g., thermostability at 75° C. or higher). The frequency distribution computing module 212 identifies amino acids that are more frequent in high-performing protein sequences such as with positive Delta T and penalize low-performing protein sequences such as with negative Delta T.

In an embodiment, each protein sequence is a string of amino acid residues such as 20 naturally occurring amino acids, and the frequency distribution computing module 212 constructs a position-wise amino acid count matrix. The matrix has dimensions PXA, where P is the number of positions in the protein sequence, and A is the number of possible amino acids such as 20. For each position P, the frequency distribution computing module 212 counts the occurrences of each amino acid across all N protein sequences, normalizing the counts to obtain a frequency distribution. For example, if at position 2, the amino acid methionine (M) appears in 100 out of 1,000 sequences, the frequency is 0.1 (10%). Further, the frequency distribution computing module 212 scales the amino acid counts at each position by the corresponding sequence's delta value, emphasizing contributions from high-performing protein sequences. For example, if methionine (M) at position 2 appears in a sequence with a delta value of +5, the count is multiplied by a positive factor (e.g., +5), increasing influence in the frequency distribution. Conversely, if methionine (M) appears at position 2 in a sequence with a delta value of −5, the count is multiplied by a negative factor, reducing influence in the frequency distribution.

In an embodiment, the updating module 214 may be configured to update the PSSM by combining the scaled token-level attribution scores and the scaled position-wise amino acid frequency distribution. The updating is performed using a normalization function to maintain the PSSM as a probability distribution. The updating module 214 may include applying a learning rate to stabilize the combination of the scaled token-level attribution scores and the scaled position-wise amino acid frequency distribution. The updating module 214 integrates the attribution scores and correlational patterns from the frequency distribution to refine the PSSM, biasing the inverse folding model toward sampling protein sequences with enhanced target properties in subsequent iterations. The updating module 214 employs a normalization function, such as a softmax function, to ensure that the PSSM remains a valid probability distribution, with values for each amino acid at each position summing to 1. Further, the updating module 214 includes applying a learning rate to stabilize the combination of the scaled token-level attribution scores and the scaled position-wise amino acid frequency distribution, controlling the magnitude of updates to prevent overfitting and ensure gradual convergence toward an optimized PSSM.

In an embodiment, the updating module 214 aggregates the scaled attribution scores and frequency distributions to produce a single updated matrix. To ensure stable updates and prevent overfitting, the updating module 214 applies a learning rate to the combined updated matrix. The learning rate may be a scalar value such as 0.01 to 0.1, which controls the magnitude of the update applied to the existing PSSM, allowing gradual refinement. The updating module 214 further applies a normalization function to the updated PSSM values. The combination of the attribution scores and frequency distributions may result in values outside the range [0, 1] or with arbitrary scales (e.g., 7230, −4200). The softmax function normalizes the values for each position across all possible amino acids in the protein sequence.

In an embodiment, the updating module 214 may be configured to mask chains in the protein sequences to optimize only targeted regions of the protein. The PSSM is updated only for the masked regions. The masking process involves designating certain segments of the protein sequence, such as individual chains, domains, or specific residues, to be excluded from optimization, restricting modifications to the unmasked, targeted regions. The PSSM, which biases the inverse folding model's sampling, is updated exclusively for the positions corresponding to the unmasked regions, while the PSSM entries for masked regions remain unchanged. The selective updating ensures that the optimization process focuses on improving the target property in specific parts of the protein sequence, such as an active site or a functional domain, without altering other regions that may be critical for structural stability or other functionalities.

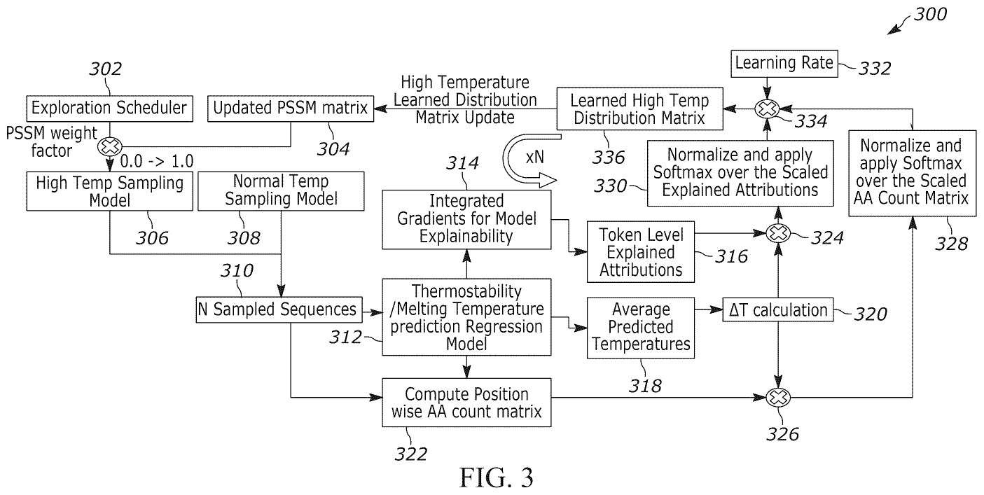

FIG. 3 illustrates a block diagram of a system architecture 300 for explainable optimization of protein sequence using inverse folding model, in accordance with an example embodiment. FIG. 3 is explained in conjunction with the FIGS. 1 and 2. The system architecture 300 refines sequence sampling for protein design such as thermostability by biasing the sampling distribution based on interpretable model signals from Integrated Gradients and amino acid distributions instead of directly altering model weights, avoiding catastrophic forgetting, maintains structural fidelity, and accelerates convergence.

The system architecture 300 may include an explorer scheduler 302 which may control how strongly the PSSM matrix influences the inverse folding model. The explorer scheduler 302 may implement as a fixed policy such as periodic schedule, cosine decay or the learnable policy network trained via Reinforcement Learning such as the PPO and the A2C. the explore schedule 302 may output a PSSM Weight Factor (w) ranging from 0 (pure exploration) to 1 (pure exploitation). Further, the explorer scheduler 302 may help escape local optima by occasionally lowering the influence of the learned PSSM matrix, promoting broader sequence space exploration.

Further, the system architecture 300 may include an updated PSSM Matrix 304 which may be a learnable matrix that biases the output of the inverse folding model. The PSSM Matrix may be initialized either randomly or using the distribution from the baseline model. The PSSM matrix is iteratively updated using feedback from attribution scores and correlation signals. Further, the PSSM matrix may encourage amino acids to increase the target property.

The system architecture 300 may include a high-temperature sampling model 306 which may be a generative model such as the ProteinMPNN or the HyperMPNN that samples sequences using the PSSM matrix bias. The high-temperature sampling model 306 may operate at high temperature (low confidence/sharpness) settings to increase diversity. Further, the high-temperature sampling model 306 may explore and then focus on high-performing areas of the protein space.

Further, the system architecture 300 may include a normal temperature sampling model 308 which may be a baseline generative model without the PSSM matrix bias. Further, the normal temperature sampling model 308 may produce protein sequences using the learned distribution over amino acids. Further, the normal temperature sampling model 308 may be used for comparison and contrast to measure how PSSM matrix bias affects protein sequence quality.

The system architecture 300 may include a N sampled sequences 310 which may be the protein sequences sets generated from the high-temperature sampling model 306 and the normal temperature sampling model 308. The N sampled sequences 310 are then scored, explained, and analysed to update the biasing strategy.

The system architecture 300 may include a thermostability/melting temperature prediction regression model 312 which predict the score of each generated sequence (e.g., Tm). The thermostability/melting temperature prediction regression model 312 may receive each amino acid sequence and returns a scalar prediction (e.g., 75° C.). Further, the scores are used to compute average batch temperature and delta (ΔT).

In an embodiment, the system architecture 300 may include an integrated gradients for model explainability 314 which explains the output of the predictor in a token-wise manner. For the protein sequence and the predicted property value, the integrated gradients for model explainability 314 assigns attribution scores to each token (amino acid). Higher attribution scores indicate that the amino acid positively contributes to thermostability, and negative attribution scores suggest deleterious effects.

The system architecture 300 may include a token level explained attribution 316 may be a tensor with same shape as the input protein structure. The token level explained attribution 316 may multiply the attribution score by delta T to reinforce directionality, higher score sequences reinforce good amino acids and lower score sequences penalize bad amino acids.

The system architecture 300 may include an average predicted temperature module 318 may establish the batch level benchmark. Further, the system architecture 300 may include a delta T calculation model 320 which measures deviation of each protein sequence from the batch average temperature. The delta T calculation model 320 scales the attribution scores and amino acid counts to reflect the contextual importance.

The system architecture 300 may include a compute position-wise amino acid count distribution 322 may create a correlation-based signal. For each position, the compute position-wise amino acid count distribution 322 counts how frequently each amino acid appears in high or low scoring sequences.

In an embodiment, the system architecture 300 may include a multiplication block 324 which may increase the learning signal for amino acid in protein sequences with positive delta T. The system architecture 300 may include a multiplication block 326 which may apply an exponential scaling to emphasize extremes while reducing influence of near-mean protein sequences, highlighting the amino acid positions where frequency shifts are statistically significant.

The system architecture 300 may include a normalized scaled amino acid count matrix module 328 to ensure the distribution matrix becomes a valid probability distribution. The normalize scaled amino acid count matrix module 328 maintains interpretability and enables application as a PSSM matrix.

Further, the system architecture 300 may include a normalize scaled attribution matrix module 330 which may process the integrated gradients attribution matrix. The normalized scaled attribution matrix module 330 may generate a probability matrix reflecting causality-based attribution.

The system architecture 300 may include a learning rate module 332 which controls the updating of the PSSM. The learning rate may be static or adaptively tuned based on attribution variance or reward trajectory. The learning rate module 332 may stabilize learning of the PSSM, preventing overshooting or excessive changes to the PSSM biasing matrix.

The system architecture 300 may include a multiplication block 334 which multiplies normalized attribution matrix with the learning rate, resulting in the incremental update applied to the PSSM. Further, the system architecture 300 may include a high-temperature distribution matrix module 336 which outputs the new PSSM matrix used in the next sampling iteration. The high-temperature distribution matrix module 336 encodes both statistical shifts such as correlation and causal effects such as explainable AI, reflecting where sampling should be focused to improve target protein properties.

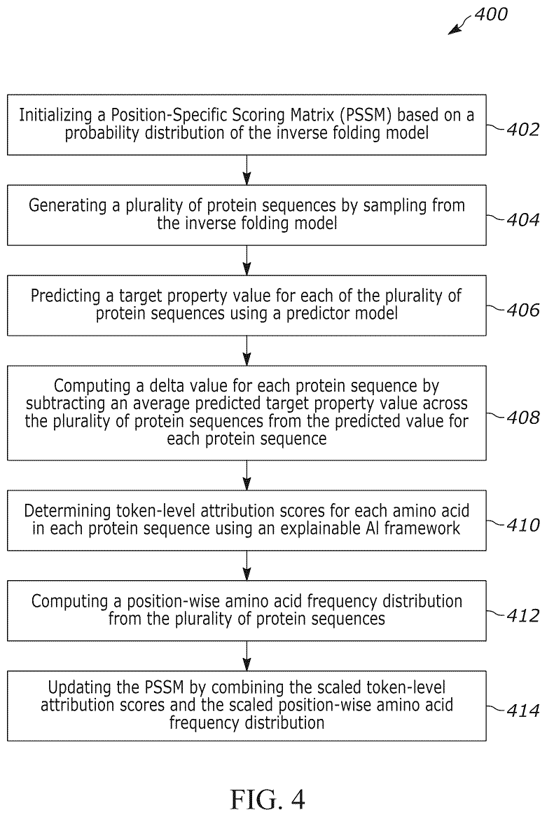

FIG. 4 illustrates a flow diagram of a method 400 for explainable optimization of protein sequence using inverse folding model, in accordance with an example embodiment. FIG. 4 is explained in conjunction with the FIGS. 1, 2 and 3. It will be understood that each block of the flow diagram of the method 400 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 106 of the computing device 102, employing an embodiment of the present disclosure and executed by a processor 104. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

At step 402, the method 400 may include initializing a Position-Specific Scoring Matrix (PSSM) based on a probability distribution of the inverse folding model. The inverse folding model generates protein sequences from a target protein structure. The inverse folding model may be a graph neural network-based model such as the ProteinMPNN and the HyperMPNN. The inverse folding model generates protein sequences conditioned on a target protein backbone structure by learning a conditional probability distribution over amino acids that fold into the specified structure. The PSSM is initialized using the inverse folding model's prior probability distribution or a random distribution, encoding position-specific probabilities or weights for each amino acid.

The method 400, at step 404, may include generating a plurality of protein sequences by sampling from the inverse folding model. The sampling is biased by applying the PSSM to the inverse folding model's output probabilities. The plurality of protein sequences is generated by using the inverse folding model, which takes a protein's 3D structure (backbone) as input and predicts which amino acid sequences are likely to fold into that structure. To guide the process toward generating sequences with better properties (like higher stability), the PSSM is applied to the inverse folding model's output probabilities. The PSSM matrix gives more weight to amino acids that are believed to improve the protein's performance. The inverse folding model then uses the adjusted probabilities to sample multiple sequences, each representing a possible protein variant that fits the desired structural and functional criteria.

At step 406, the method 400 may include predicting a target property value for each of the plurality of protein sequences using a predictor model. The target property may refer to a specific functional or structural characteristic of the protein that is being optimized, for example, thermostability, solubility, binding affinity, or enzymatic activity. Further, the predictor model is a trained machine learning model that receives a protein sequence (amino acid chain) as input and outputs a scalar value corresponding to the estimated property of interest. For example, the predictor model may predict the temperature at which the protein remains folded and functional. Each protein sequence is individually passed through the predictor model outputting a predicted value of the target property for that protein sequence.

At step 408, the method 400 may include computing a delta value for each protein sequence by subtracting an average predicted target property value across the plurality of protein sequences from the predicted value for each protein sequence. The delta value reflects how much better or worse a particular protein sequence performs relative to the batch mean. A positive delta indicates that the protein sequence outperforms the average and may contain beneficial features, while a negative delta suggests below-average performance.

The method 400, at step 410, may include determining token-level attribution scores for each amino acid in each protein sequence using an explainable AI framework. The attribution scores indicates a contribution of each amino acid to the predicted target property value. Each amino acid within the protein sequence is treated as a “token”, and the XAI framework is used to assess the relative importance or contribution of each token to the predicted target property value. For example, if the target property is thermostability, the XAI framework may identify which amino acids are responsible for increasing or decreasing the predicted melting temperature of the protein sequence. Each attribution score corresponds to a specific amino acid in a specific protein sequence and quantifies influence on the overall predicted property value.

At step 412, the method 400 may include computing a position-wise amino acid frequency distribution from the plurality of protein sequences. The frequency distribution is scaled by the delta value of each protein sequence to emphasize frequencies in the protein sequences with higher predicted target property value. The position-wise amino acid frequency distribution captures statistical correlation patterns across the plurality of protein sequences, providing insight into which amino acids commonly occur at specific positions in protein sequences associated with high predicted target property values. For each position in the protein sequence (e.g., position 1, 2, . . . , L for a sequence of length L), the method 400 determines how frequently each of the amino acids appears across all sequences in the protein sequence, resulting in a position-wise amino acid frequency matrix, where each entry represents the occurrence of a particular amino acid at a particular protein sequence position.

Further, each protein sequence's frequency contribution is weighted by its corresponding delta value, such that sequences with higher predicted target property values such as positive delta contribute more strongly to the frequency distribution, while protein sequences with lower values such as negative delta contribute less or may be penalized. In some embodiments, the scaling may be further enhanced using exponential weighting functions to amplify differences between high and low performer protein sequences.

At step 414, the method 400 may include updating the PSSM by combining the scaled token-level attribution scores and the scaled position-wise amino acid frequency distribution. The updating is performed using a normalization function to maintain the PSSM as a probability distribution. To update the PSSM, the method 400 may aggregates the attribution scores and the frequency distribution either by weighted summation or other combination techniques. Further, the method 400 applies a normalization function, such as a softmax, min-max normalization, or z-score transformation, to ensure that each row of the PSSM such as each sequence position remains a valid probability distribution over the amino acids.

In an exemplary embodiment, consider a scenario where a biotechnology company seeks to engineer a variant of an existing enzyme that retains its activity at temperatures exceeding 80° C. to improve efficiency in biomass degradation for bioethanol production. Using the present disclosure, the company inputs the enzyme's 3D structure into an inverse folding model and employs the disclosed optimization loop guided by explainable AI and statistical frequency analysis to generate a refined set of amino acid sequences predicted to enhance thermostability. The Position-Specific Scoring Matrix (PSSM) is iteratively updated using token-level attribution scores and position-wise amino acid frequencies scaled by predicted melting temperatures, allowing the system to sample sequences more likely to remain stable at high temperatures. The resulting variants may be experimentally validated, drastically reducing the number of candidates required for wet-lab screening, saving time, cost, and resources, while ensuring a rational, interpretable, and efficient protein engineering workflow.

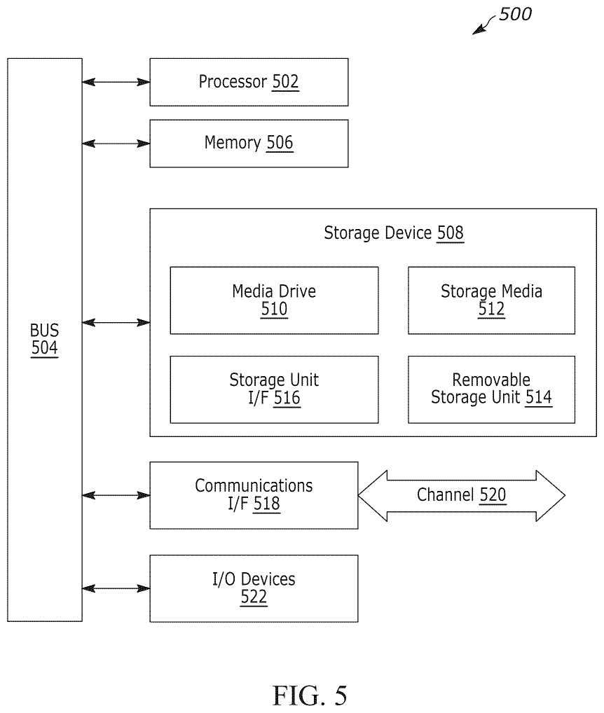

The disclosed methods and systems may be executed on a conventional or general-purpose computing system, such as a personal computer (PC) or server. Referring to FIG. 5, an exemplary computing system 500 is illustrated, which may implement processing functionality for various embodiments (e.g., as a SIMD device, client device, server device, or one or more processors). Those skilled in the art will recognize that other computing systems or architectures may also be used to implement the invention. The computing system 500 may represent a user device, such as a desktop, laptop, mobile phone, personal entertainment device, DVR, or any other special or general-purpose computing device appropriate for a given application or environment.

The computing system 500 may include one or more processors, such as processor 502, implemented using a general-purpose or specialized processing engine, such as a microprocessor, microcontroller, or other control logic. In some embodiments, processor 502 may be an AI processor, implemented as a Tensor Processing Unit (TPU), graphical processing unit (GPU), or custom-programmable solution, such as a Field-Programmable Gate Array (FPGA).

The computing system 500 may further include memory 506 (e.g., Random Access Memory (RAM) or other dynamic memory) for storing instructions and information to be executed by processor 502. Memory 506 may also store temporary variables or intermediate information during execution. Additionally, the computing system 500 may include a read-only memory (ROM) or other static storage device connected to bus 504 for storing static information and instructions for processor 502.

Storage devices 508 may also be included in computing system 500, consisting of, for example, a media drive 510 and a removable storage interface. Media drive 510 may support fixed or removable storage media, such as hard disk drives, floppy drives, magnetic tape drives, SD card ports, USB ports, optical disk drives (e.g., CD or DVD drives), or other media. Storage media 512 may include hard disks, magnetic tapes, flash drives, or other media that can be read and written to by media drive 510. Storage media 512 may store computer-readable software or data.

Alternatively, storage devices 508 may include other means for loading computer programs or data into computing system 500, such as removable storage unit 514 and interface 516, program cartridges, removable memory (e.g., flash memory), memory slots, and similar storage units and interfaces.

Computing system 500 may also include a communications interface 518 to transfer software and data between external devices 112 and system 100. Examples include network interfaces (e.g., Ethernet), communication ports (e.g., USB, micro-USB), Near Field Communication (NFC), and other protocols. The signals transferred via communications interface 518 may include electronic, electromagnetic, optical, or other forms of transmission through channel 520, which may utilize wireless mediums, fibre optics, wires, or cables.

Computing system 500 may also include Input/Output (I/O) devices 522, such as a display, keypad, microphone, speakers, vibration motors, LED indicators, etc., allowing user interaction and feedback. The term “computer-readable medium” may refer to any storage medium used, such as memory 506, storage devices 508, removable storage unit 514, or signal(s) on channel 520. Such media may store sequences of instructions, or “computer program code,” which, when executed, enable computing system 500 to perform the methods and functions described in embodiments of the invention.

In embodiments where elements are implemented in software, the software may be stored on a computer-readable medium and loaded into computing system 500 via removable storage unit 514, media drive 510, or communications interface 518. When executed by processor 502, this control logic (e.g., software instructions or computer program code) causes processor 502 to perform the invention's functions as described.

As will be appreciated by those skilled in the art, the techniques described in the various embodiments discussed above are not routine, or conventional, or well understood in the art. The techniques discussed above provide for innovative solutions to address the challenges associated with explainable optimization of protein sequence using inverse folding model. The disclosed techniques offer several advantages over the existing methods:

Faster Convergence: The present disclosure significantly reduces the number of sequences needed to identify high-performing candidates (e.g., from 50 million to 200,000 sequences for thermostability optimization), achieving convergence in fewer iterations compared to traditional Reinforcement Learning (RL) or oversampling methods.

Training Stability: By keeping the inverse folding model weights fixed and updating only the PSSM matrix, the present disclosure avoids catastrophic forgetting, ensuring generated sequences remain structurally valid without requiring computationally expensive fold validation.

Explainability: The present disclosure utilizes explainable Artificial Intelligence (XAI) (e.g., Integrated Gradients) to provide token-level attribution scores, revealing which amino acids contribute positively or negatively to the target property such as thermostability, enabling rational and interpretable protein design.

Efficient Search Space Reduction: The present disclosure narrows the vast protein sequence search space by orders of magnitude by biasing sampling toward high-performing regions using a refined PSSM matrix, reducing computational resources required.

Targeted Optimization: The present disclosure supports optimization of specific protein regions, chains, or residues by masking sequences, allowing precise updates to the PSSM matrix for targeted areas, increasing flexibility in protein design.

Gradient-Free Optimization: The present disclosure uses a gradient-free approach by learning a PSSM matrix rather than fine-tuning model weights, reducing computational complexity and reliance on gradient-based updates.

The disclosed techniques offer several applications including:

Thermostability Optimization: The present disclosure designs proteins that remain stable and functional at high temperatures, useful for industrial enzymes in processes requiring elevated temperatures, such as biofuel production or chemical synthesis.

Solubility Enhancement: The present disclosure optimizes protein sequences to improve solubility, applicable in pharmaceutical development for producing soluble therapeutic proteins or in industrial applications, where solubility impacts protein performance.

Protein-Protein Binding Affinity Improvement: The present disclosure enhances the binding affinity of proteins for specific targets, relevant for developing biologics, such as antibodies or protein-based drugs, in therapeutic applications.

Rational protein design for drug development: The present disclosure facilitates the rational design of proteins with improved therapeutic properties, such as enhanced stability or binding specificity, for use in drug discovery and development.

Industrial Biotechnology: The present disclosure engineers proteins for industrial processes, such as developing enzymes with improved performance under harsh conditions (e.g., high pH, temperature, or solvent exposure) for applications in food processing, textiles, or detergent.

Synthetic biology: The present disclosure creates novel proteins with tailored functionalities for synthetic biology applications, such as designing proteins for new metabolic pathways or bioengineering systems.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

We claim:

1. A computer-implemented method (400) for explainable optimization of protein sequence using inverse folding model, the method (400) comprising:
initializing (402) a Position-Specific Scoring Matrix (PSSM) based on a probability distribution of the inverse folding model or from a database of similar proteins, wherein the inverse folding model generates protein sequences from a target protein structure;
generating (404) a plurality of protein sequences by sampling from the inverse folding model, wherein the sampling is biased by applying the PSSM to the inverse folding model's output probabilities;
predicting (406) a target property value for each of the plurality of protein sequences using a predictor model;
computing (408) a delta value for each protein sequence by subtracting an average predicted target property value across the plurality of protein sequences from the predicted value for each protein sequence;
determining (410) token-level attribution scores for each amino acid in each protein sequence using an explainable AI framework, wherein the attribution scores indicates a contribution of each amino acid to the predicted target property value;
computing (412) a position-wise amino acid frequency distribution from the plurality of protein sequences, wherein the frequency distribution is scaled by the delta value of each protein sequence to emphasize frequencies in the protein sequences with higher predicted target property value; and
updating (414) the PSSM by combining the scaled token-level attribution scores and the scaled position-wise amino acid frequency distribution, wherein the updating is performed using a normalization function to maintain the PSSM as a probability distribution.

2. The computer-implemented method (400) of claim 1, wherein the inverse folding model comprises a graph neural network-based model comprising ProteinMPNN and HyperMPNN.

3. The computer-implemented method (400) of claim 1, wherein the explainable AI framework is selected from the group consisting of Integrated Gradients, SHapley Additive explanations (SHAP), Local Interpretable Model-agnostic Explanations (LIME), and attention-based techniques.

4. The computer-implemented method (400) of claim 1, wherein the predictor model is configured to predict one or more of a thermostability, a melting temperature, a solubility, a catalytic activity, and a binding affinity of the protein.

5. The computer-implemented method (400) of claim 1, wherein updating the PSSM further comprises applying a learning rate to stabilize the combination of the scaled token-level attribution scores and the scaled position-wise amino acid frequency distribution.

6. The computer-implemented method of claim 1, further comprising applying a weight factor to the PSSM, wherein the weight factor controls a degree of bias applied to the inverse folding model's output probabilities.

7. The computer-implemented method (400) of claim 6, wherein the weight factor is dynamically adjusted using a scheduler to balance exploration and exploitation, wherein the scheduler being selected from the group consisting of a cosine scheduler, a fixed interval scheduler, and a reinforcement learning policy network.

8. The computer-implemented method (400) of claim 1, further comprising masking chains in the protein sequences to optimize only targeted regions of the protein, wherein the PSSM is updated only for the masked regions.

9. A computer-implemented system (100) for explainable optimization of protein sequence using inverse folding model, the computer-implemented system (100) comprising: one or more computer processors (104), one or more computer readable memories (106), one or more computer readable storage devices, and program instructions stored on the one or more computer readable storage devices for execution by the one or more computer processors (104) via the one or more computer readable memories (106), the program instructions comprising:
initializing a Position-Specific Scoring Matrix (PSSM) based on a probability distribution of the inverse folding model, wherein the inverse folding model generates protein sequences from a target protein structure;
generating a plurality of protein sequences by sampling from the inverse folding model, wherein the sampling is biased by applying the PSSM to the inverse folding model's output probabilities;
predicting a target property value for each of the plurality of protein sequences using a predictor model;
computing a delta value for each protein sequence by subtracting an average predicted target property value across the plurality of protein sequences from the predicted value for each protein sequence;
determining token-level attribution scores for each amino acid in each protein sequence using an explainable AI framework, wherein the attribution scores indicates a contribution of each amino acid to the predicted target property value;
computing a position-wise amino acid frequency distribution from the plurality of protein sequences, wherein the frequency distribution is scaled by the delta value of each protein sequence to emphasize frequencies in the protein sequences with higher predicted target property value; and
updating the PSSM by combining the scaled token-level attribution scores and the scaled position-wise amino acid frequency distribution, wherein the updating is performed using a normalization function to maintain the PSSM as a probability distribution.

10. The computer-implemented system (100) of claim 9, wherein the inverse folding model comprises a graph neural network-based model comprising ProteinMPNN and HyperMPNN.

11. The computer-implemented system (100) of claim 9, wherein the explainable AI framework is selected from the group consisting of Integrated Gradients, SHapley Additive explanations (SHAP), Local Interpretable Model-agnostic Explanations (LIME), and attention-based techniques.

12. The computer-implemented system (100) of claim 9, wherein the predictor model is configured to predict one or more of a thermostability, a melting temperature, a solubility, a catalytic activity, and a binding affinity of the protein.

13. The computer-implemented system (100) of claim 9, wherein updating the PSSM further comprises applying a learning rate to stabilize the combination of the scaled token-level attribution scores and the scaled position-wise amino acid frequency distribution.

14. The computer-implemented system (100) of claim 9, further comprising applying a weight factor to the PSSM, wherein the weight factor controls a degree of bias applied to the inverse folding model's output probabilities.

15. The computer-implemented system (100) of claim 14, wherein the weight factor is dynamically adjusted using a scheduler to balance exploration and exploitation, wherein the scheduler being selected from the group consisting of a cosine scheduler, a fixed interval scheduler, and a reinforcement learning policy network.

16. The computer-implemented system (100) of claim 9, further comprising masking chains in the protein sequences to optimize only targeted regions of the protein, wherein the PSSM is updated only for the masked regions.

17. A non-transitory computer-readable storage medium having stored thereon computer executable instruction which when executed by one or more processors (104), cause the one or more processors (104) to carry out operations for explainable optimization of protein sequence using inverse folding model, the operations comprising:
initializing a Position-Specific Scoring Matrix (PSSM) based on a probability distribution of the inverse folding model, wherein the inverse folding model generates protein sequences from a target protein structure;
generating a plurality of protein sequences by sampling from the inverse folding model, wherein the sampling is biased by applying the PSSM to the inverse folding model's output probabilities;
predicting a target property value for each of the plurality of protein sequences using a predictor model;
computing a delta value for each protein sequence by subtracting an average predicted target property value across the plurality of protein sequences from the predicted value for each protein sequence;
determining token-level attribution scores for each amino acid in each protein sequence using an explainable AI framework, wherein the attribution scores indicates a contribution of each amino acid to the predicted target property value;
computing a position-wise amino acid frequency distribution from the plurality of protein sequences, wherein the frequency distribution is scaled by the delta value of each protein sequence to emphasize frequencies in the protein sequences with higher predicted target property value; and
updating the PSSM by combining the scaled token-level attribution scores and the scaled position-wise amino acid frequency distribution, wherein the updating is performed using a normalization function to maintain the PSSM as a probability distribution.

18. The non-transitory computer-readable storage medium of claim 17, wherein the inverse folding model comprises a graph neural network-based model comprising ProteinMPNN and HyperMPNN.

19. The non-transitory computer-readable storage medium of claim 17, wherein the explainable AI framework is selected from the group consisting of Integrated Gradients, SHapley Additive explanations (SHAP), Local Interpretable Model-agnostic Explanations (LIME), and attention-based techniques.

20. The non-transitory computer-readable storage medium of claim 17, wherein the predictor model is configured to predict one or more of a thermostability, a melting temperature, a solubility, a catalytic activity, and a binding affinity of the protein.
